SEQUENCE LISTING (5 sequences)

SEQ ID NO: 1 - protein, 7 aa, unidentified
TLAWNWK

SEQ ID NO: 2 - protein, 14 aa, unidentified
PNWGRGRPSSFIDT

SEQ ID NO: 3 - protein, 11 aa, unidentified
YNSGYGIADTN

SEQ ID NO: 4 - protein, 111 aa, unidentified
MSDTALIFTLAWNVKQLAFDYTPNWGRGRPSSFIDTVTFPTVLTDKAYTYRVVVSGKDLGVRPSYAVESDGSQKINFLEYNSGYGIADTNTIQVYVIDPDTGNNFIVAQWN

SEQ ID NO: 5 - protein, 140 aa, synthetic construct
EAEAEFMSDTALIFTLAWNVKQLAFDYTPNWGRGRPSSFIDTVTFPTVLTDKAYTYRVVVSGKDLGVRPSYAVESDGSQKINFLEYNSGYGIADTNTIQVYVIDPDTGNNFIVAQWNYLEQKLISEEDLNSAVDHHHHHH

(19) United States
(12) Patent Application Publication
(10) Pub. No.: US 2023/0233633 A1
(43) Pub. Date: Jul. 27, 2023

(54) METHOD FOR TREATING PD-L1 EXPRESSING CANCER

(71) Applicant: National Yang Ming Chiao Tung University, Taipei (TW)

(72) Inventor: Tung-Yi Lin, Taipei (TW)

(21) Appl. No.: 17/937,850

(22) Filed: Oct. 4, 2022

Related U.S. Application Data
(60) Provisional application No. 63/303,523, filed on Jan. 27, 2022.

(51) Int. Cl. A61K 36/074; A61K 38/16; A61K 39/395; A61P 35/00

(52) U.S. Cl. CPC A61K 36/074; A61K 38/16; A61K 39/3955; A61P 35/00; A61K 2039/505

(57) ABSTRACT

Provided is a method for treating cancers expressing immune checkpoint protein PD-L1. Also provided is a method for reducing PD-L1 expression of cancer cells by administering to a subject in need thereof an effective amount of an immunomodulatory protein of Ganoderma, a recombinant thereof, or a fungal immunomodulatory protein of a similar structure.

### TECHNICAL FIELD

The present disclosure relates to a method for treating a cancer, particularly to a method for treating a cancer expressing immune checkpoint protein programmed death-ligand 1 (PD-L1). The disclosure also relate to a method for reducing PD-L1 expression in a cancer cell.

### SEQUENCE LISTING

The present application is being filed along with a Sequence Listing in electronic format. The Sequence Listing is provided as a file entitled 21057.0039USU1.XML, created on Sep. 16, 2022, which is 5,578 bytes (about 6 KB) in size. The information in the electronic format of Sequence Listing is incorporated herein by reference in its entirety.

### BACKGROUND

Cancer has been a major global health problem due to its high morbidity and mortality. While many chemotherapy agents have been developed and applied in clinical practice, their application is limited due to toxic adverse effects and relatively poor tolerability. During the last decades, cancer immunotherapy has been accompanied with promising results. It is noted that immunotherapy has achieved therapeutic efficacy in a variety of malignancies, including non-small cell lung cancer, renal cell carcinoma, melanoma, breast cancer and colorectal cancer.

Programmed death-1 (PD-1) is a cell surface receptor that functions as a T cell checkpoint and plays a central role in regulating T cell exhaustion. Binding of PD-1 to its ligand, programmed death-ligand 1 (PD-L1), activates downstream signaling pathways and inhibits T cell activation.

Until now, there is no therapeutic product directing to cancer cells’ PD-L1 downregulation and degradation. Therefore, there remains an unmet need to develop an effective pharmaceutic agent with safety and tolerability to treat cancers.

### SUMMARY

In view of the foregoing, the present disclosure provides a method for suppressing immune checkpoint protein PD-L1 expression, or inducing degradation of PD-L1 in a cancer cell, comprising administering an effective amount of an immunomodulatory protein derived from Ganoderma, a recombinant thereof, or a fungal immunomodulatory protein of a similar structure as an active agent to a subject suffering from malignant neoplasm or cancer.

In some embodiments, the immunomodulatory protein or the recombinant thereof is derived from Ganoderma lucidum, Ganoderma tsugae, Ganoderma microsporum, Ganoderma applanatum, Ganoderma japonicum, Ganoderma astum, Ganoderma atrum, or Ganoderma sinensis. In other embodiments, the immunomodulatory protein is LZ-8 derived from Ganoderma lucidum, FIP-gts derived from Ganoderma tsugae, GMI derived from Ganoderma microsporum, FIP-gap derived from Ganoderma applanatum, FIP-gja derived from Ganoderma japonicum, FIP-gas derived from Ganoderma astum, FIP-gat derived from Ganoderma atrum, or FIP-gsi derived from Ganoderma sinensis or any recombinant thereof.

In some embodiments, the cancer is melanoma, or carcinoma of the head and neck, brain, glioblastoma multiforme, nervous system, thyroid, thymus, esophagus, stomach, lung, breast, gastrointestinal tract, colorectum, liver, pancreas, kidney, adrenal cortex, genitourinary system, prostate, bladder, urothelium, uterus, cervix, ovary, skin, or hematologic malignancy. In at least one embodiment, the cancer is small cell lung cancer (SCLC), non-small cell lung cancer (NSCLC), squamous carcinoma of lung or adenocarcinoma of lung. In other embodiments, the cancer is primary cancer or secondary cancer. In further embodiments, the cancer is localized cancer, regional cancer, advanced cancer, or metastatic cancer. In at least one embodiment, the cancer is solid tumor or non-solid tumor. In other embodiments, the cancer is sarcoma, carcinoma, lymphoma, or leukemia.

In at least one embodiment, the pharmaceutical composition is locally or systemically delivered to the subject, and the subject can be a mammal, for example, a human.

In at least one embodiment, the pharmaceutical composition is administered orally or rectally. In other embodiments, the pharmaceutical composition is administered parenterally.

In at least one embodiment, the pharmaceutical composition can be administered orally or rectally through an appropriate formulation with carriers and excipients to form tablets, pills, capsules, liquids, gels, syrups, slurries, suspensions and the like. In other embodiments, the pharmaceutical composition can be administered by an inhaler to the respiratory tract for local or systemic treatment of cancers.

In at least one embodiment, the parenteral administration is intravenous, drip, subcutaneous, intraperitoneal or intramuscular injection, or intrathecal or intraventricular administration.

In some further embodiments, the method of the present disclosure further comprises administering a second active agent. The second active agent is used with the immunomodulatory protein sequentially, concurrently or separately.

The present disclosure also provides an aqueous formulation for parenteral administration comprising the said immunomodulatory protein as an active agent. In some embodiments, the amount of the active agent in the formulation ranges from about 0.5 mg/mL to about 150 mg/mL.

The present disclosure further provides a use of a pharmaceutical composition in the manufacture of a medicament for treating cancer and/or inhibiting immune checkpoint protein PD-L1 expression in a cancer cell, and the pharmaceutical composition comprises an effective amount of an immunomodulatory protein of Ganoderma, a recombinant thereof, or a fungal immunomodulatory protein of a similar structure and the pharmaceutically acceptable carrier thereof as mentioned above.

### BRIEF DESCRIPTION OF THE DRAWINGS

The present disclosure can be more fully understood by reading the following descriptions of the embodiments, with reference made to the accompanying drawings.

FIG. 1 illustrates the inhibitory effect of GMI of the present disclosure on glycosylated PD-L1 expression in various lung cancer cells including H1975, CL1-5, A549, and LLC-1. Cells were treated with GMI (0 to 0.6 µM) for 3 hours, followed by Western blotting to detect the expression of glycosylated PD-L1 and phosphorylated GSK3β. α-Tubulin was used as the internal control.

FIGS. 2A and 2B show the PD-L1 degradation induced by GMI in lung cancer cells. FIG. 2A illustrates the analysis of the half-life of PD-L1 protein levels in 200 µg/mL cycloheximide treated H1975 and CL1-5 cells with or without GMI (0.6 µM) for 0 to 12 h. FIG. 2B shows the PD-L1 expression levels in lung cancer cells pretreated with DMSO (vehicle control) or MG132 (proteasome inhibitor, 10 µM) for 30 min and then treated with GMI (0.6 µM) for 24 h. The PD-L1 expression levels in the indicated experiments were analyzed via Western blotting. α-Tubulin was used as the internal control.

FIGS. 3A and 3B show the effect of lithium chloride (LiCl) upon GMI-downregulated PD-L1 levels in lung cancer cells. H1975 (FIG. 3A) and CL1-5 (FIG. 3B) cells were pretreated with PBS (vehicle control) and LiCl (GSK3β inhibitor, 25 mM) for 30 min and then treated with GMI (0.6 µM) for 24 h. The levels of PD-L1 in indicated experiments were analyzed via Western blotting. α-Tubulin was used as the internal control. Quantification of the intensities of the bands of PD-L1 in experiments is the representative of three separate determinations by ImageJ. The data are presented as the mean ± SD; error bars indicate SDs. Significant differences are shown (* * * P < 0.001, ** P < 0.01, * P < 0.05, compared between the indicated groups).

FIGS. 4A and 4B show the effect of GMI in downregulation of PD-L1 expression in lung tumor lesions of LLC1-bearing mouse. Five individual experiments are shown. Each bar represents the mean ± SD. Significant differences are shown (***P < 0.001 compared to the control group).

### DETAILED DESCRIPTION OF THE EMBODIMENTS

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the exemplary methods and materials are now described. All publications mentioned herein are incorporated herein by reference.

As used in this disclosure, the singular forms “a,” “an,” and “the” include plural referents unless expressly and unequivocally limited to one referent. The term “or” is used interchangeably with the term “and/or” unless the context clearly indicates otherwise.

As used herein, the term “comprising” or “comprises” is used in reference to compositions, methods, and respective component(s) thereof, which are included in the present disclosure, yet open to the inclusion of unspecified elements or steps, whether essential or not.

As used herein, the term “an effective amount” is the quantity of an active agent which achieves a clinical outcome when the compound is administered to a subject. For example, when an active agent of the disclosure is administered to a subject with a cancer, a “clinical outcome” includes reduction in tumor mass, reduction in metastasis, reduction in the severity of the symptoms associated with the cancer and/or increase in the longevity of the subject. The effective amount may vary, as recognized by those skilled in the art, depending on routes of administration, excipient usage, the possibility of co-usage with other therapeutic treatment, and the condition to be treated.

As used herein, the term “about” generally referring to the numerical value meant to encompass variations of ±20%, ±10%, ±5%, ±1%, ±0.5%, or ±0.1% from a given value or range. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. Alternatively, the term “about” means within an acceptable standard error of the mean when considered by one of ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

The numeral ranges used herein are inclusive and combinable, any numeral value that falls within the numeral scope herein could be taken as a maximum or minimum value to derive the sub-ranges therefrom. For example, it should be understood that the numeral range “0.01 mg to 10 mg” comprises any sub-ranges between the minimum value of 0.01 mg to the maximum value of 10 mg, such as the sub-ranges from 0.01 mg to 5 mg, from 1 mg to 10 mg, from 5 mg to 8 mg and so on. In addition, a plurality of numeral values used herein can be optionally selected as maximum and minimum values to derive numerical ranges. For instance, the numerical ranges of 1.0 mg/mL to 20 mg/mL, 1.0 mg/mL to 100 mg/mL, and 20 mg/mL to 100 mg/mL can be derived from the numeral values of 1.0 mg/mL, 20 mg/mL, and 100 mg/mL.

According to the disclosure, the terms “treatment,” “treating” and the like are used herein to generally mean obtaining a desired pharmacologic or physiologic effect. The effect may be prophylactic in terms of completely or partially preventing a condition, appearance, disease or symptom and/or may be therapeutic in terms of a partial or complete cure for a condition and/or adverse effect attributable to a condition or disease. The term “treatment” as used herein covers any treatment of a condition, disease or undesirable appearance in a mammal, e.g., a human, and includes: (a) preventing the disease (e.g., cancer), condition (pain) or appearance (e.g., visible tumors) from occurring in a subject which may be predisposed to it but has not yet been observed or diagnosed as having it; (b) inhibiting the disease, condition or symptom, i.e., causing regression of a condition or symptom; and (c) relieving the disease, condition or symptom, i.e., causing regression of a condition or symptom.

As used herein, the terms “patient” and “subject” are used interchangeably. The term “subject” means a human or an animal. Examples of the subject include, but are not limited to, human, monkey, mice, rat, woodchuck, ferret, rabbit, hamster, cow, horse, pig, deer, dog, cat, fox, wolf, chicken, emu, ostrich, and fish. In some embodiments of the present disclosure, the subject is a mammal, e.g., a primate such as a human.

As used herein, the term “administering” or “administration” refers to the placement of an active agent into a subj ect by a method or route which results in at least partial localization of the active agent at a desired site to produce a desired effect. The active agent described herein may be administered by any appropriate route known in the art. For example, the pharmaceutical composition of the present disclosure is administered to the subject by oral administration.

As used herein, the term “recombinant” may refer to the alteration of genetic material by human intervention. For example, recombinant may refer to the manipulation of DNA or RNA in a cell or virus or an expression vector by molecular biology (recombinant DNA technology) methods, including cloning and recombination. Recombinant may also refer to manipulation of DNA or RNA in a cell or virus by random or directed mutagenesis. A “recombinant” nucleic acid can be described with reference to how it differs from a naturally occurring counterpart (the “wild-type”). A recombinant protein may refer to a protein expressed by recombinant DNA technology. A recombinant protein has a similar amino acid sequence and maintains the same activity or function as its parental protein.

Lingzhi, an herbal mushroom, used in traditional Chinese medicine for at least 2,000 years, is a species complex that encompasses several fungal species of the genus Ganoderma, most commonly Ganoderma lucidum, Ganoderma tsugae, and Ganoderma sichuanense, which are closely related. Many therapeutic effects have been reported of Lingzhi, such as immunomodulatory, anti-tumor, hepato-protective, antioxidant, and cholesterol-lowering effects (Jinn et al., 2006, Biosci. Biotechnol. Biochem., 70, 2627-2634). Most of these therapeutic effects are attributed to triterpenoids, polysaccharides, and glycoproteins (Boh et al., 2007, Biotechnol. Annu. Rev., 13, 265-301; Jinn et al., 2006, Biosci. Biotechnol. Biochem., 70, 2627-2634). A glycoprotein class in Lingzhi named fungal immunomodulatory proteins (FIPs) has recently been identified. So far, at least 5 FIPs have been isolated, i.e., LZ-8, (Ganoderma lucidum), FIP-gts (Ganoderma tsugae), FIP-gja (Ganoderma sinensis) and GMI (Ganoderma microsporum) (Ko et al., 1995, Eur. J. Biochem., 228, 244-249).

Abnormally high PD-L1 expression on tumor cells and antigen-presenting cells in the tumor microenvironment mediates tumor immune escape, and thus development of anti-PD-1/PD-L1 antibodies is a hot topic in cancer immunotherapy. Antibody blockade of the PD-1/PD-L1 pathway has elicited durable antitumor responses in the therapy of a broad spectrum of cancers.

Constitutive expression of PD-L 1 in tumor cells is linked to dysregulation of oncogenic or tumor suppressor gene signaling pathways, through activation of abnormal transcription factors or genomic aberrations or gene amplifications. PD-L1 overexpression has also been associated with more aggressive pathological features and poorer prognosis in certain cancers. Therefore, in addition to the efforts devoted to the search of anti-cancer agents through PD-1 or PD-L1 antibody blockade, downregulation of PD-L1 expression may also play a role in cancer therapy and become another strategy in treating cancer.

The present disclosure provides an immunomodulatory protein of Ganoderma microsporum (GMI) that is effective in suppressing immune checkpoint protein PD-L1 expression of cancer cells and treating cancers. For example, the GMI induces PD-L1 degradation in a cancer cell through the proteasomal degradation system. The GMI activates GSK3β, thereby inducing PD-L1 degradation in a cancer cell. The PD-L1 expression in a lung cancer-bearing mouse is downregulated by GMI. In addition, the combination of GMI and anti-PD-1 antibody suppresses tumor growth in the lung cancer-bearing mouse. These findings suggest GMI’s efficacy in cancer immunotherapy.

In one aspect, the method of the present disclosure for treating cancer comprises suppression of tumor growth, progression, or recurrence. In another aspect, the method of the present disclosure for treating cancer comprises prevention of cancer development.

In one aspect, the Ganoderma immunomodulatory protein, a recombinant thereof, or a fungal immunomodulatory protein of a similar structure in combination with an anti-cancer drug(s) provides an effect in the treatment and/or prevention of cancer. In another aspect, the present disclosure provides a pharmaceutical composition, which comprises a Ganoderma immunomodulatory protein, a recombinant thereof, or a fungal immunomodulatory protein of a similar structure, and an anti-cancer agent. The composition exhibits a synergistic effect in treating and/or preventing cancer.

In one embodiment, the Ganoderma immunomodulatory protein, a recombinant thereof, or a fungal immunomodulatory protein of a similar structure is derived from Ganoderma lucidum, Ganoderma tsugae, Ganoderma microsporum, Ganoderma applanatum, Ganoderma japonicum, Ganoderma astum, Ganoderma atrum, or Ganoderma sinensis. For example, the immunomodulatory protein is LZ-8 derived from Ganoderma lucidum, FIP-gts derived from Ganoderma tsugae, GMI derived from Ganoderma microsporum, FIP-gap derived from Ganoderma applanatum, FIP-gja derived from Ganoderma japonicum, FIP-gas derived from Ganoderma astum, FIP-gat derived from Ganoderma atrum, or FIP-gsi derived from Ganoderma sinensis or a recombinant thereof. In some embodiments, the immunomodulatory protein is derived from Ganoderma microsporum (GMI) or Ganoderma lucidum (LZ-8).

According to this disclosure, the Ganoderma immunomodulatory protein (e.g., GMI), a recombinant thereof, or a fungal immunomodulatory protein of a similar structure has the amino acid sequence of: (1) TLAWNWK (SEQ ID NO: 1), (2) PNWGRGRPSSFIDT (SEQ ID NO: 2), and (3) YNSGYGIADTN (SEQ ID NO: 3), or the amino acid sequence of

MSDTALIFTLAWNVKQLAFDYTPNWGRGRPSSFIDTVTFPTVLTD
KAYTY RVVVSGKDLGVRPSYAVESDGSQKINFLEYNSGYGIADTN
TIQVYVIDPD TGNNFIVAQWN (SEQ ID NO: 4)

, or the amino acid sequence of

EAEAEFMSDTALIFTLAWNVKQLAFDYTPNWGRGRPSSFIDTVTFP
TVLT DKAYTYRVVVSGKDLGVRPSYAVESDGSQKINFLEYNSGYGI
ADTNTIQV YVIDPDTGNNFIVAQWNYLEQKLISEEDLNSAVDHHHH
HH (SEQ ID NO: 5).

In at least one embodiment, the pharmaceutical composition comprising GMI of the present disclosure can be used in combination with radiotherapy and/or chemotherapy. In some embodiments, the pharmaceutical composition can be used in combination with radiotherapy, chemotherapy and/or immunotherapy.

In at least one embodiment, the pharmaceutical composition comprising GMI of the present disclosure can be combined with an anti-cancer agent for combination therapy in cancer. The Ganoderma immunomodulatory protein, the recombinant thereof, or the fungal immunomodulatory protein of a similar structure in the present disclosure can further be combined with an anti-cancer agent as a pharmaceutical composition. For example, the present disclosure may provide a pharmaceutical composition comprising Ganoderma immunomodulatory protein, a recombinant thereof, or a fungal immunomodulatory protein of a similar structure and an anti-cancer agent, and said pharmaceutical composition can treat and/or prevent cancers. The cancer may be melanoma, carcinoma of the head and neck, brain, glioblastoma multiforme, nervous system, thyroid, thymus, esophagus, stomach, lung, breast, gastrointestinal tract, colorectum, liver, pancreas, kidney, adrenal cortex, genitourinary system, prostate, bladder, urothelium, uterus, cervix, ovary, skin, or hematologic malignancy. For example, the cancer is lung cancer (e.g., non-small cell lung carcinoma (NSCLC) and squamous cell carcinomas of the lung), head and neck cancer, breast cancer, ovarian cancer, prostate cancer, gastric carcinoma, cervical cancer, esophageal carcinoma, bladder cancer, brain cancer, liver cancer, or colon cancer. In at least one embodiment, the composition of the present disclosure exhibits a synergistic efficacy.

In some embodiments, the effective amount of the active agent of the pharmaceutical composition is about 0.01 mg to about 10 mg protein per 70 kg body weight for a human. In some further embodiments, the effective amount of the active agent is about 1.0 mg to about 5 mg protein per 70 kg body weight for a human. In other embodiments, the effective amount of the active agent used in the method of the present disclosure is about 10 mg to about 100 mg protein per 70 kg human body weight, e.g., about 20 mg to about 80 mg protein per 70 kg body weight, about 20 mg to about 50 mg protein per 70 kg body weight, about 25 mg to about 50 mg protein per 70 kg body weight, about 30 mg to about 50 mg protein per 70 kg body weight, about 35 mg to about 50 mg protein per 70 kg body weight, or about 30 mg to about 40 mg protein per 70 kg body weight.

In some embodiments, the effective amount of the active agent of the pharmaceutical composition used in the method of the present disclosure is about 0.001 mg to about 0.1 mg protein per 1 kg body weight, e.g., about 0.0125 mg protein per 1 kg body weight, or about 0.05 mg protein per 1 kg body weight.

In other embodiments of the present disclosure, the effective amount of the active agent of the pharmaceutical composition may be administered to a subject 1 to 4 times per day, 1 to 4 times per week, or 1 to 4 times per month. In further embodiments of the present disclosure, the effective amount of the active agent of the pharmaceutical composition may be administered to a subject every 1, 2, 3, 4, 5, 6, or 7 days.

In another aspect, the present disclosure provides an aqueous formulation for parenteral administration comprising a Ganoderma immunomodulatory protein, a recombinant thereof or a fungal immunomodulatory protein of a similar structure as an active agent. In at least one embodiment, the amount of the active agent in the formulation ranges from about 0.5 mg/mL to about 150 mg/mL, e.g., about 1.0 mg/mL to about 100 mg/mL, about 1.0 mg/mL to about 80 mg/mL, about 1.0 mg/mL to about 60 mg/mL, about 1.0 mg/mL to about 40 mg/mL, about 1.0 mg/mL to about 20 mg/mL, about 5 mg/mL to about 150 mg/mL, about 5 mg/mL to about 100 mg/mL, about 10 mg/mL to about 150 mg/mL, or about 10 mg/mL to about 100 mg/mL.

The parenteral formulations may be in unit dose form in ampoules, small volume parenteral (SVP) vials, large volume parenterals (LVPs), pre-filled syringes, small volume infusion or in multi-dose containers. The formulations are suspensions or solutions and may contain formulatory agents such as preserving, wetting, buffering, emulsifying or suspending, stabilizing and/or dispersing agents. Alternatively, the ratio, type, and varieties of the ingredients, active and inactive, are studied to reach an optimal balance, before use with a suitable vehicle, e.g., sterile, pyrogen-free water. Some embodiments are contemplated that are substantially free of buffers, stabilizers, and/or preservatives, while still preserving the formulation’s chemical stability, pH value, and product sterility.

Parenteral formulations are typically aqueous solutions which may contain excipients such as salts, carbohydrates and buffering agents (e.g., at a pH of from 3 to 9.5). Additional embodiments are substantially buffer free.

In some embodiments, the parenteral formulation comprises a single dose pH adjusted solution having an effective amount of an active agent for treating a cancer; a tonicity agent for adjusting osmolality to about physiological osmolality; optional pH adjusting reagents; and sterile water for injection.

In at least one embodiment, the parenteral formulations contain a solution of an active agent in an aqueous solvent combined with pH adjusting agents and at least one isotonicity agent. A water-insoluble inert gas may be carefully bubbled through the solvent to remove oxygen from the medium. Optionally, the formulations contain at least one preservative and/or at least one solubility enhancing agent and/or at least one stabilizing agent. In some embodiments, the formulation is substantially free of stabilizing agents and preservatives.

Tonicity agents are sometimes present. The term “tonicity agent” refers to a pharmaceutically acceptable excipient that makes the solution compatible with blood. Suitable tonicity agents include glycerin, lactose, mannitol, dextrose, sodium chloride, sodium sulfate, sorbitol and the like. In some embodiments, tonicity agents include mannitol, sorbitol, lactose and sodium chloride and any combinations thereof. The tonicity agent is added to the injectable to achieve substantially physiological osmolality for injection.

Hypertonic and hypotonic solutions both present complications and undesirable effects when injected. The parenteral formulations described herein are isotonic to minimize or avoid such effects. Since osmolality is the measure of particles in a solution, every component added to the injectable affects the osmolality, and thus adjusting to a final osmolality is complicated. For example, when also adjusting the pH, as addition of the tonicity agent may affect pH, addition of the pH adjusting reagents will affect tonicity.

Optional pH adjusting reagents include acids and bases, such as dilute HCl and NaOH. An acid may be added to lower the pH, while the base is added to raise the pH. In some instances, one or both an acid and a base may be used. In some embodiments, the pH adjusting reagents are chosen to complement the tonicity agent to provide similar ions when in solution. For example, when NaCl is used as a tonicity agent, HCl and/or NaOH may be used as the pH adjusting reagents.

The tonicity agent, such as NaCl, is employed to achieve an isotonic solution. Isotonic solutions for injection have an osmolality roughly equivalent to physiological osmolality. Other concentrations of NaCl may result in either undesirable hypertonic or hypotonic solutions.

Additional components, such as active agents, excipients, diluents, buffers, preservatives, etc. may be employed, so long as the parenteral formulation remains isotonic and stable. Any suitable additional active agent could optionally be incorporated into the parenteral formulation.

In some embodiments, the method and formulation described herein optionally further comprises a second active agent. The second active agent may be used with the immunomodulatory protein of the present disclosure sequentially, concurrently or separately. In at least one embodiment, the second active agent include, but is not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cisplatin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypernycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithineklerriene; emitefur; epirubicin; epristeride; erlotinib; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorubicin hydrochloride; forfenimex; formestane; fostriecin; fotemustine; gadolinium texaphyrin; gallium nitrate; galocitabine; ganirelix; gefitinib; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; 4-ipomeanol; iroplact; irsogladine; isobengazole; isohomohalicondrin B; itasetron; jasplakinolide; kahalalide F; lamellarin-N triacetate; lanreotide; lapatinib; leinamycin; lenograstim; lentinan sulfate; leptolstatin; letrozole; leukemia inhibiting factor; leukocyte alpha interferon; leuprolide plus estrogen plus progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitors; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody; human chorionic gonadotrophin; monophosphoryl lipid A plus mycobacterial cell wall skeleton; mopidamol; multiple drug resistance gene inhibitors; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone plus pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O⁶-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulators; protein kinase C inhibitors; microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; rarnosetran; ras farnesyl protein transferase inhibitors; ras inhibitors; ras-GAP inhibitor; demethylated retelliptine; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonerrnin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Additional anti-cancer drugs may be 5-fluorouracil and leucovorin.

According to the present disclosure, the anti-cancer agent can be a therapeutic antibody, including, but not limiting to, Herceptin (Trastuzumab) (Genentech, California), which is a humanized anti-HER2 monoclonal antibody for the treatment of patients with metastatic breast cancer; Reopro (abciximab) (Centocor), which is an anti-glycoprotein IIb/IIIa receptor on the platelets for the prevention of clot formation; Zenapax (daclizumab) (Roche Pharmaceuticals, Switzerland), which is an immunosuppressive, humanized anti-CD25 monoclonal antibody for the prevention of acute renal allograft rejection; Panorex, which is a murine anti-17-IA cell surface antigen IgG2a antibody (Glaxo Wellcome/Centocor); BEC2, which is a murine anti-idiotype (GD3 epitope) IgG antibody (ImClone System); IMC-C225, which is a chimeric anti-EGFR IgG antibody (ImClone System); Vitaxin, which is a humanized anti-alpha.Vbeta.3 integrin antibody (Applied Molecular Evolution/MedImmune); Campath 1H/LDP-03, which is a humanized anti-CD52 IgG1 antibody (Leukosite); Smart M195, which is a humanized anti-CD33 IgG antibody (Protein Design Lab/Kanebo); Rituxan, which is a chimeric anti-CD20 IgG1 antibody (IDEC Pharm/Genentech, Roche/Zettyaku); Lymphocide, which is a humanized anti-CD22 IgG antibody (Immunomedics); Lymphocide Y-90 (Immunomedics); Lymphoscan (Tc-99m-labeled; radioimaging; Immunomedics); Nuvion (against CD3; Protein Design Labs); CM3, which is a humanized anti-ICAM3 antibody (ICOS Pharm); IDEC-114, which is a primatied anti-CD80 antibody (IDEC Pharm/Mitsubishi); Zevalin, which is a radiolabelled murine anti-CD20 antibody (IDEC/Schering AG); IDEC-131, which is a humanized anti-CD40L antibody (IDEC/Eisai); IDEC-151, which is a primatized anti-CD4 antibody (IDEC); IDEC-152, which is a primatized anti-CD23 antibody (IDEC/Seikagaku); SMART anti-CD3, which is a humanized anti-CD3 IgG (Protein Design Lab); 5G1.1, which is a humanized anti-complement factor 5 (C5) antibody (Alexion Pharm); D2E7, which is a humanized anti-TNF-alpha antibody (CAT/BASF); CDP870, which is a humanized anti-TNF-alpha Fab fragment (Celltech); IDEC-151, which is a primatized anti-CD4 IgG1 antibody (IDEC Pharm/SmithKline Beecham); MDX-CD4, which is a human anti-CD4 IgG antibody (Medarex(Eisai/Genmab)); CD20-sreptdavidin (+biotin-yttrium 90; NeoRx); CDP571, which is a humanized anti-TNF-alpha IgG4 antibody (Celltech); LDP-02, which is a humanized anti-alpha-4-beta-7 antibody (LeukoSite/Genentech); OrthoClone OKT4A, which is a humanized anti-CD4 IgG antibody (Ortho Biotech); Antova, which is a humanized anti-CD40L IgG antibody (Biogen); Antegren, which is a humanized anti-VLA-4 IgG antibody (Elan); and CAT-152, which is a human anti-TGF-beta 2 antibody (Cambridge Ab Tech).

In a further embodiment, the anti-cancer agent can be selected from the group consisting of cisplatin, gefitinib, lapatinib and erlotinib.

According to the present disclosure, the anti-cancer agent is an anti-PD-1 antibody or an anti-PD-L1 antibody. In at least one embodiment, the anti-PD-1 antibody or the anti-PD-L1 antibody is Yervoy (ipilimumab), Keytruda (pembrolizumab) or Opdivo (nivolumab).

Without further elaboration, it is believed that one skilled in the art can utilize the present disclosure to its fullest extent on the basis of the preceding description. The following examples are, therefore, to be construed as merely illustrative and not a limitation of the scope of the present disclosure in any way.

### EXAMPLES

The immunomodulatory protein derived from Ganoderma microsporum (hereinafter referred to as “GMI”) used in the examples was manufactured by Mycomagic Biotechnology Co., Ltd., according to the method described in U.S. Pat. No. 7,601,808 and has an amino acid sequence of

MSDTALIFTLAWNVKQLAFDYTPNWGRGRPSSFIDTVTFPTVLTDK
AYTY RVVVSGKDLGVRPSYAVESDGSQKINFLEYNSGYGIADTNTI
QVYVIDPD TGNNFIVAQWN (SEQ ID NO: 4).

The human NSCLC adenocarcinoma cell lines A549 and CL1-5 were obtained from American Type Culture Collection (ATCC, USA) and Dr. Pan-Chyr Yang (NTU, Taiwan), respectively. H1975 cell line bought from Blossom Biotechnologies Inc. (Taipei, Taiwan). Lewis lung carcinoma cell line (LLC1) was purchased from the Bioresource Collection and Research Center (BCRC, Hsinchu, Taiwan). A549 and LLC1 cells were cultured in Dulbecco’s modified Eagle’s medium (DMEM, GIBCO/Life Technologies) supplemented with 10% fetal bovine serum. H1975 cell was cultured in Roswell Park Memorial Institute medium 1640 (RPMI medium 1640, GIBCO/Life Technologies) supplemented with 10% FBS, 2 g/L of NaHCO₃ and 100 units/mL each of penicillin and streptomycin (Biological Industries, Cromwell, CT, USA) at 37° C. All of the adherent cells were detached by incubation with trypsin-EDTA (Invitrogen, Co., Carlsbad, CA).

Statistical differences between the experimental groups were determined using a t-test in GraphPad Prism8. A vale of P < 0.05 indicates a statistically significant result. All data are expressed as mean ± standard deviation (SD).

### Example 1: GMI Reduces PD-L1 Expression

Lung cancer cells including H1975, CL1-5, A549, and LLC1 were treated with GMI (0 to 0.6 µM) for 3 hours followed by Western blotting to detect the expression of PD-L1 and phosphorylated GSK3β. α-Tubulin was used as the internal control.

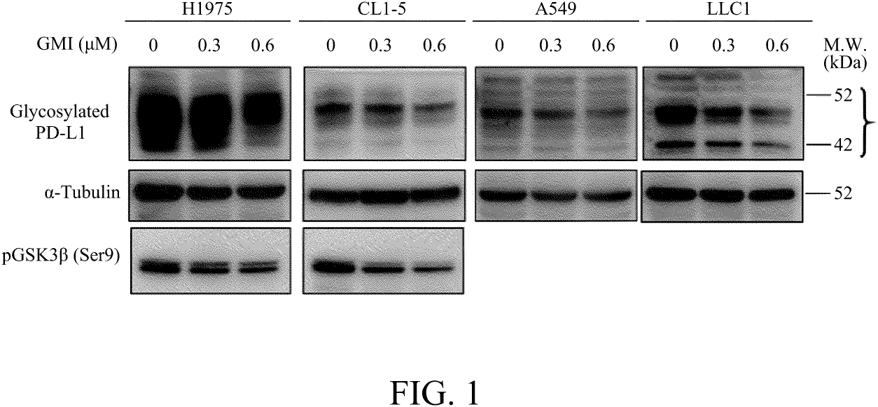

As shown in FIG. 1, the result showed that GMI effectively reduced mature (glycosylated) PD-L1 expression in various lung cancer cell lines in a short duration of time, suggesting that GMI is effective in suppressing PD-L1 expression, and is useful in cancer immunotherapy.

### Example 2: GMI Induces PD-L1 Degradation

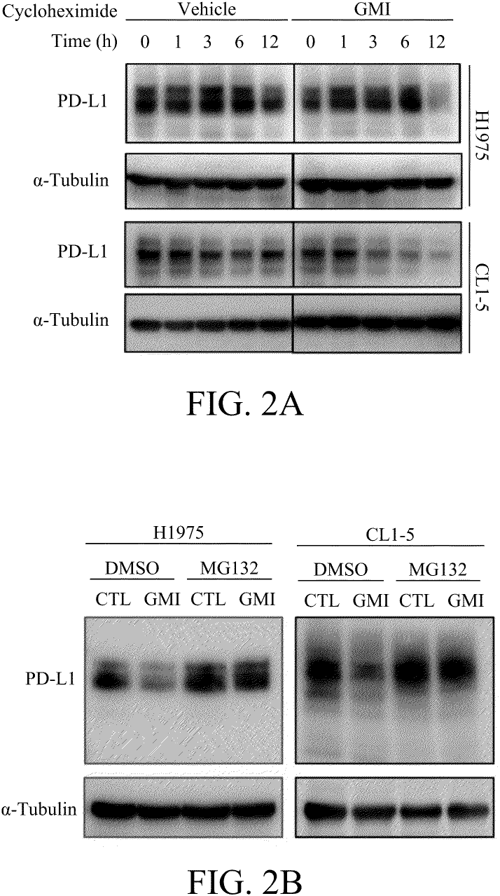

The half-life of PD-L1 in H1975 and CL1-5 cells was examined upon cycloheximide (CHX) treatment. In detail, the half-life of PD-L 1 protein levels in 200 µg/mL cycloheximide treated H1975 and CL1-5 cells with or without GMI (0.6 µM) for 0 to 12 hours was analyzed. As shown in FIG. 2A, the half-life of PD-L1 in lung cancer cells was much shorter when the cells were exposed to CHX combined with GMI. These results suggested that GMI induces and accelerates PD-L1 degradation.

It has been previously documented that PD-L1 degradation depends on proteasomal system (Cell Death Dis. 2020; 11(11):955). Therefore, the proteasome inhibitor, MG132, was used to abolish the activity of proteasome. It was found that MG132 recovered the GMIreduced PD-L1 expression in lung cancer cells. Specifically, cells were pretreated with DMSO (vehicle control) or MG132 (proteasome inhibitor, 10 µM) for 30 min and then treated with GMI (0.6 µM) for 24 h. The levels of PD-L1 in indicated experiments were analyzed via Western blotting. α-Tubulin was used as the internal control. As shown in FIG. 2B, the results indicated that the induction of proteasomal degradation system is involved in the GMI-induced degradation of PD-L1.

### Example 3: GMI Induces PD-L1 Degradation

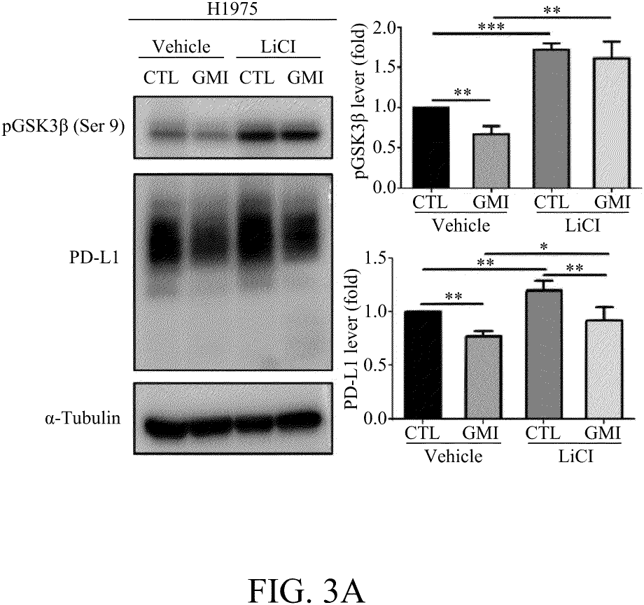

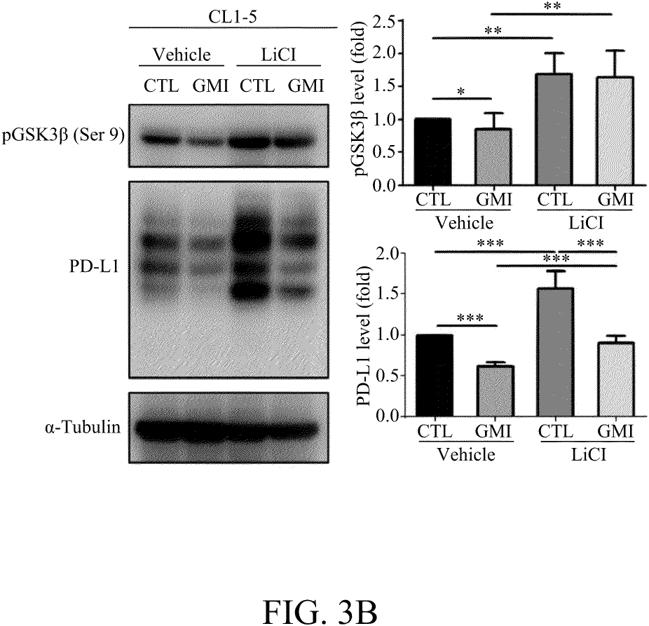

To further investigate the involvement of GMI-inhibited intracellular signaling in PD-L1 turnover, the glycogen synthase kinase 3β (GSK3β) activity in regulation of PD-L1 protein levels has been examined. It was shown that GSK3β interacts with PD-L1 and induces phosphorylation-dependent proteasome degradation of PD-L1 by β-TrCP (Nat. Commun. 2016; 7:12632). GSK3β activity is inhibited through AKT-mediated phosphorylation of GSK3β (Nature. 1995; 378(6559):785-9). As shown in FIGS. 1, 3A and 3B, the analysis examining the effects of GMI on phosphorylation of GSK3β (ser 9) demonstrated that GMI reduced phosphorylation of GSK3β, suggesting that GMI-induced PD-L1 degradation depends on activation of GSK3β.

H1975 and CL1-5 cells were pretreated with PBS (vehicle control) and LiCl (GSK3β inhibitor, 25 mM) for 30 min and then treated with GMI (0.6 µM) for 24 hours. The levels of PD-L1 in indicated experiments were analyzed via Western blotting, as shown in FIGS. 3A and 3B. α-Tubulin was used as the internal control. Quantification of the band intensities of PD-L1 in experiments is the representative of three separate determinations by ImageJ. The data are presented as the mean ± SD; error bars indicate SDs. Significant differences are shown (* * * P < 0.001, compared with the control group). Briefly, by using GSK3β inhibitor (LiCl), it is found that LiCl counteracted GMI-induced PD-L1 degradation. Taking together, these results indicated that GMI induces the GSK3β-mediated PD-L1 degradation.

### Example 4: GMI Downregulates PD-L1 Expression in Lung Tumor Lesions of LLC1-Bearing Mouse

Mice were subcutaneously inoculated with LLC1 cells and then randomly divided into two groups. Beginning one day after inoculation, the mice were treated with GMI (5 mg/kg) via i.p. injections at 4-day intervals. The control (CTL) group received equal volumes of sterile PBS. PD-L1 protein levels were assessed in the tumor lesions using Western blot analysis. Five individual experiments are shown. Each bar represents the mean ± SD. Significant differences are shown (* * *P < 0.001 compared to the control group).

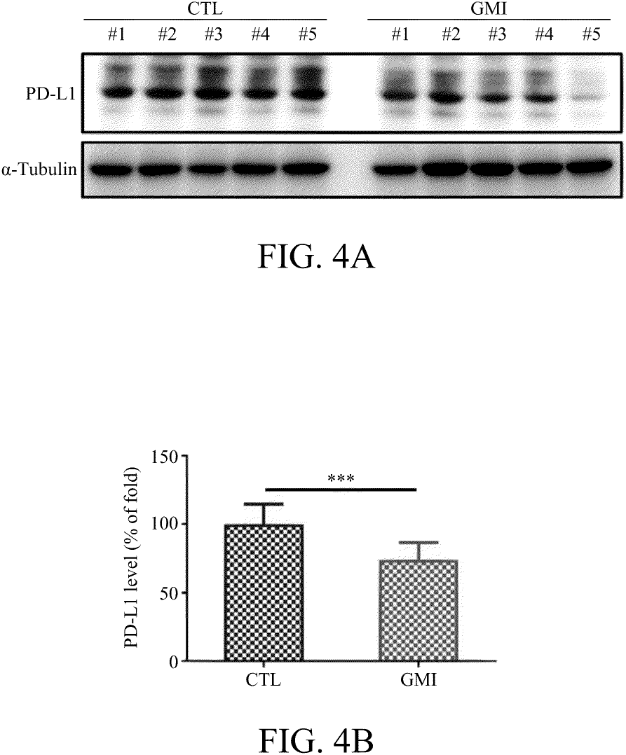

As shown in FIGS. 4A and 4B, in LLC1-bearing mice, GMI treatment significantly decreases PD-L1 level over CTL.

### Example 5: Combination of GMI and anti-PD-1 Antibody Suppresses Tumor Growth in LLC1-Bearing Mouse

In this example, the effect of GMI in combination with an anti-PD-1 antibody was determined in the LLC1-bearing mice. The results suggest that the co-treatment of GMI and anti-PD-1 antibody has a significantly synergistic effect in suppressing tumor growth in the LLC1-bearing mice.

While some of the embodiments of the present disclosure have been described in detail above, it is, however, possible for those of ordinary skill in the art to make various modifications and changes to the embodiments shown without substantially departing from the teaching and advantages of the present disclosure. Such modifications and changes are thus encompassed in the scope of the present disclosure as set forth in the appended claims.

What is claimed is:

1. A method for inhibiting immune checkpoint protein PD-L1 expression in a cancer cell, comprising contacting the cancer cell with an effective amount of an immunomodulatory protein of Ganoderma, a recombinant thereof, or a fungal immunomodulatory protein of a similar structure.

2. The method of claim 1, wherein the immunomodulatory protein of Ganoderma or the recombinant thereof has:
an amino acid sequences of (1) TLAWNWK (SEQ ID NO: 1), (2) PNWGRGRPSSFIDT (SEQ ID NO: 2), and (3) YNSGYGIADTN (SEQ ID NO: 3);
an amino acid sequence of MSDTALIFTLAWNVKQLAFDYTPNWGRGRPSSFIDTVTFPTVLTDKAYTY RVVVSGKDLGVRPSYAVESDGSQKINFLEYNSGYGIADTNTIQVYVIDPD TGNNFIVAQWN (SEQ ID NO: 4); or
an amino acid sequence of EAEAEFMSDTALIFTLAWNVKQLAFDYTPNWGRGRPSSFIDTVTFPTVLT DKAYTYRVVVSGKDLGVRPSYAVESDGSQKINFLEYNSGYGIADTNTIQV YVIDPDTGNNFIVAQWNYLEQKLISEEDLNSAVDHHHHHH (SEQ ID NO: 5).

3. The method of claim 1, wherein the inhibition of immune checkpoint protein PD-L1 of the cancer cell is induction of PD-L1 degradation.

4. The method of claim 1, wherein the Ganoderma is Ganoderma lucidum, Ganoderma tsugae, Ganoderma microsporum, Ganoderma applanatum, Ganoderma japonicum, Ganoderma astum, Ganoderma atrum, or Ganoderma sinensis.

5. The method of claim 1, wherein the immunomodulatory protein is selected from the group consisting of LZ-8, FIP-gts, GMI, FIP-gap, FIP-gja, FIP-gas, FIP-gat, FIP-gsi, a recombinant LZ-8, a recombinant FIP-gts, a recombinant GMI, a recombinant FIP-gap, a recombinant FIP-gja, a recombinant FIP-gas, a recombinant FIP-gat, a recombinant FIP-gsi, and any combination thereof.

6. A method for treating cancer in a subject in need thereof, comprising administering to the subject with an effective amount of an immunomodulatory protein of Ganoderma, a recombinant thereof, or a fungal immunomodulatory protein of a similar structure, wherein the cancer comprises a cancer cell expressing PD-L1.

7. The method of claim 6, wherein the immunomodulatory protein of Ganoderma or the recombinant thereof has:
an amino acid sequence of (1) TLAWNWK (SEQ ID NO: 1), (2) PNWGRGRPSSFIDT (SEQ ID NO: 2), and (3) YNSGYGIADTN (SEQ ID NO: 3);
an amino acid sequence of MSDTALIFTLAWNVKQLAFDYTPNWGRGRPSSFIDTVTFPTVLTDKAYTY RVVVSGKDLGVRPSYAVESDGSQKINFLEYNSGYGIADTNTIQVYVIDPD TGNNFIVAQWN (SEQ ID NO: 4); or
an amino acid sequence of EAEAEFMSDTALIFTLAWNVKQLAFDYTPNWGRGRPSSFIDTVTFPTVLT DKAYTYRVVVSGKDLGVRPSYAVESDGSQKINFLEYNSGYGIADTNTIQV YVIDPDTGNNFIVAQWNYLEQKLISEEDLNSAVDHHHHHH (SEQ ID NO: 5).

8. The method of claim 6, wherein the Ganoderma is Ganoderma lucidum, Ganoderma tsugae, Ganoderma microsporum, Ganoderma applanatum, Ganoderma japonicum, Ganoderma astum, Ganoderma atrum, or Ganoderma sinensis.

9. The method of claim 6, wherein the immunomodulatory protein is selected from the group consisting of LZ-8, FIP-gts, GMI, FIP-gap, FIP-gja, FIP-gas, FIP-gat, FIP-gsi, a recombinant LZ-8, a recombinant FIP-gts, a recombinant GMI, a recombinant FIP-gap, a recombinant FIP-gja, a recombinant FIP-gas, a recombinant FIP-gat, a recombinant FIP-gja, and any combination thereof.

10. The method of claim 6, wherein the cancer is selected from the group consisting of melanoma, and carcinoma of head and neck, brain, glioblastoma multiforme, nervous system, thyroid, thymus, esophagus, stomach, lung, breast, gastrointestinal tract, colorectum, liver, pancreas, kidney, adrenal cortex, genitourinary system, prostate, bladder, urothelium, uterus, cervix, ovary, skin, and hematologic malignancy.

11. The method of claim 6, wherein the immunomodulatory protein of Ganoderma is administered in combination with an anti-cancer agent.

12. The method of claim 11, wherein the anti-cancer agent is an anti-PD-1 antibody or an anti-PD-L1 antibody.

13. The method of claim 11, wherein the immunomodulatory protein of Ganoderma and the anti-cancer agent are administered simultaneously, sequentially or separately.

14. The method of claim 6, wherein the immunomodulatory protein of Ganoderma is administered in combination with radiotherapy, chemotherapy, immunotherapy or any combination thereof.

15. The method of claim 6, wherein the immunomodulatory protein of Ganoderma is administered orally or rectally.

16. The method of claim 6, wherein the immunomodulatory protein of Ganoderma is administered parenterally.

17. The method of claim 6, wherein the effective amount of the immunomodulatory protein of Ganoderma is about 0.01 mg to about 100 mg protein per 70 kg human body weight.
